(19) United States
(12) Patent Application Publication
(10) Pub. No.: US 2023/0288367 A1
(43) Pub. Date: Sep. 14, 2023

(54) DETECTION DEVICE, DETECTION METHOD, LEARNING DEVICE, AND DETECTION DEVICE MANUFACTURING METHOD

(71) Applicant: THE UNIVERSITY OF TOKYO, Tokyo (JP)

(72) Inventors: Takahisa TANAKA, Tokyo (JP); Takeaki YAJIMA, Tokyo (JP); Ken UCHIDA, Tokyo (JP)

(21) Appl. No.: 18/161,205

(22) Filed: Jan. 30, 2023

Related U.S. Application Data
(63) Continuation of application No. PCT/JP2021/027855, filed on Jul. 28, 2021.

(30) Foreign Application Priority Data
Jul. 28, 2020 (JP) 2020-127611

(51) Int. Cl. G01N 27/414; G01N 27/416

(52) U.S. Cl. CPC G01N 27/4141; G01N 27/416

(57) ABSTRACT

A detection device includes: an ion conductor; three or more electrodes that are in contact with the ion conductor; and an ammeter embodying a measurement unit that measures a potential difference between two electrodes when a fluid sample is in contact with the ion conductor or the electrode, the two electrodes being selected from the three or more electrodes in a plurality of combinations.

### BACKGROUND OF THE INVENTION

### 1. Field of the Invention

The present disclosure relates to technology for detecting a fluid and, more particularly, to a detection device, a detection method, a learning device, and a detection device manufacturing method.

### 2. Description of the Related Art

A gas component in a small amount contained in exhaled air has attracted attention as a biomarker for health conditions and diseases. For example, hydrogen is produced by intestinal bacteriological activities. It is known that hydrogen concentration in exhaled air rises from about 10 ppm before a meal to about 100 ppm after a meal (see non-patent literature 1). It is also known that ammonia concentration in exhaled air of a healthy person is about 0.32-1.08 ppm but exhaled air of patients affected with Helicobacter pylori and end-stage renal disease patients contain ammonia in higher concentration (see non-patent literatures 2 and 3).
[Non-patent literature 1] W. Shin, Analytical Bioanalytical Chem., 406, p. 3931, 2014
[Non-patent literature 2] Kearney D. et al., Dig. Dis. Sci., 47, pp. 2523-2530, 2002
[Non-patent literature 3] Davies S. et al., Kidney Int., 52, pp. 223-228, 1997

In order to use a gas component in exhaled air as a biomarker, a technology to detect a gas component in a small amount contained in a mixed gas with high precision is necessary.

### SUMMARY OF THE INVENTION

The present disclosure addresses the above-described issue, and a purpose thereof is to improve detection precision of a detection device.

A detection device according to an aspect of the present disclosure includes: an ion conductor; three or more electrodes that are in contact with the ion conductor; and a measurement unit that measures a potential difference between two electrodes when a sample fluid is in contact with the ion conductor or the electrode, the two electrodes being selected from the three or more electrodes in a plurality of combinations.

Another embodiment of the present disclosure relates to a detection method. The method includes measuring, when an ion conductor or three or more electrodes in contact with the ion conductor is in contact with a sample of fluid, a potential difference between two electrodes selected from the three or more electrodes, measurements being made a plurality of times for different combinations of two electrodes.

Still another embodiment of the present disclosure relates to a learning device. The learning device includes: a training data acquisition unit that acquires, as training data, data indicating potential differences, measured by the measurement unit, between two electrodes paired in a plurality of combinations by using a fluid for which a component is known as a training sample, from the above detection device; and a training unit that trains, by using the training data acquired by the training data acquisition unit, an estimator for estimating whether a component included in a sample of fluid is found or an amount thereof.

Still another embodiment of the present disclosure also relates to a learning device. The learning device includes: a training data acquisition unit that acquires, as training data, information relating to each of a plurality of samples and data indicating potential differences, measured by the measurement unit in the samples, between two electrodes paired in a plurality of combinations; and a training unit that categorizes or clusters the training data acquired by the training data acquisition unit.

Still another embodiment of the present disclosure relates to a detection device manufacturing method. The method includes: determining a type, composition, or surface condition of a metal constituting the three or more electrodes based on at least one of: a type and amount of a component subject to detection included in the sample; and a type and amount of a component that could be included in the sample other than the component subject to detection; and providing the three or more electrodes constituted by a metal of a type, composition, or surface condition determined so as to be in contact with the ion conductor.

Optional combinations of the aforementioned constituting elements, and implementations of the present disclosure in the form of methods, devices, systems, recording mediums, computer programs, etc. may also be practiced as additional modes of the present disclosure.

### BRIEF DESCRIPTION OF THE DRAWINGS

Embodiments will now be described, by way of example only, with reference to the accompanying drawings which are meant to be exemplary, not limiting, and wherein like elements are numbered alike in several Figures, in which:

FIG. 1 shows a configuration of a detection system according to an embodiment of the present disclosure.

FIG. 2 schematically shows a configuration of a sensor unit of the detection device according to the embodiment of the present disclosure.

FIG. 3 schematically shows the cross section of the sensor unit of the detection device according to the embodiment.

FIG. 4 shows an equivalent circuit of the sensor unit.

FIG. 5 shows an exemplary configuration of the ion conductor and the electrode of the sensor unit.

FIGS. 6A and 6B show exemplary measurement results determined by the sensor unit of an exemplary embodiment.

FIG. 7 shows exemplary measurement results determined by the sensor unit of the exemplary embodiment.

FIG. 8 shows an exemplary configuration of a component estimator for estimating the concentration of each component by referring to the measurement result determined by the sensor unit of the exemplary embodiment.

FIG. 9A to FIG. 9C show results determined by estimating the concentration of components contained in the sample gases by using the component estimator that has been trained.

FIG. 10 shows another exemplary configuration of the ion conductor and the electrodes of the sensor unit.

FIG. 11A and FIG. 11B show a result of simulation of the response by the sensor unit of the exemplary embodiment.

FIG. 12 shows another example of the ion conductor and the electrodes of the sensor unit.

FIG. 13 shows a configuration of a learning device according to the embodiment.

FIG. 14 shows a configuration of the detection device according to the embodiment.

FIG. 15 is a flowchart showing a sequence of steps of the learning method according to the embodiment.

FIG. 16 is a flowchart showing a sequence of steps of the detection method according to the embodiment.

### DETAILED DESCRIPTION OF THE INVENTION

The invention will now be described by reference to the preferred embodiments. This does not intend to limit the scope of the present invention, but to exemplify the invention.

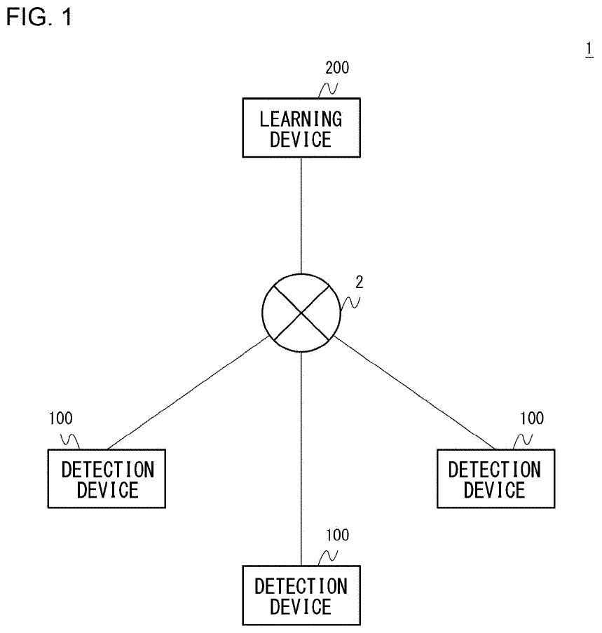

FIG. 1 shows a configuration of a detection system 1 according to an embodiment of the present disclosure. The detection system 1 is provided with a detection device 100 that detects a component subject to detection included in a sample subject to measurement, a learning device 200 that trains an estimator used in the detection device 100, and a communication network 2 connecting the detection device 100 and the learning device 200. As described later, the detection device 100 measures a potential difference between two electrodes in contact with a sample of fluid. The learning device 200 uses a measurement result acquired from the detection device 100 as training data and trains a component estimator for estimating whether a component subject to detection included in the sample of fluid is found, the amount thereof, etc. Further, the learning device 200 uses a measurement result acquired from the detection device 100 as training data to train a status estimator for estimating the health condition of a subject person or a disease that the subject is affected with, by using a component subject to detection included in the sample as a biomarker, etc. The detection device 100 uses the component estimator and the status estimator trained by the learning device 200 to estimate, from the measurement result, whether a component subject to detection is found and the amount thereof, the health condition of the subject person, etc.

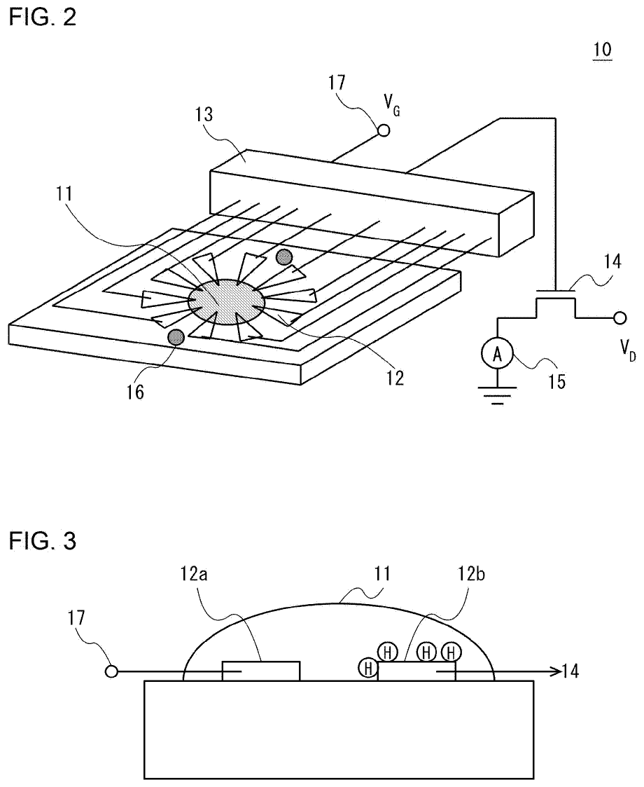

FIG. 2 schematically shows a configuration of a sensor unit 10 of the detection device according to the embodiment of the present disclosure. The sensor unit 10 is provided with an ion conductor 11, an electrode 12, a switch matrix 13, a transistor 14, an ammeter 15, a drying part 16, a measurement terminal 17, and a power source (not shown) that supplies a voltage to the drain terminal of the transistor 14. Three or more electrodes 12 are provided so as to be in contact with the ion conductor 11 and a sample of fluid common to the electrodes. The switch matrix 13 selects two electrodes from the three or more electrodes 12, connects one of the electrodes to the measurement terminal 17, and connects the other to the gate terminal of the transistor 14. For measurement, a voltage VG is applied from the power source to the measurement terminal 17, and a voltage VD is applied from the power source to the drain terminal of the transistor 14. The current that flows between the drain terminal and the source terminal of the transistor 14 is measured by the ammeter 15.

FIG. 3 schematically shows the cross section of the sensor unit 10 of the detection device according to the embodiment. The ion conductor 11 is provided to cover electrodes 12a and 12b provided on a substrate. The switch matrix 13 connects the electrode 12a to the measurement terminal 17 and connects the electrode 12b to the drain terminal of the transistor 14. On the surface of the electrode 12b made of a metal such as platinum (Pt) and rhodium (Rh), molecules of hydrogen and organic compounds contained in the sample gas could be decomposed by a catalytic reaction to produce electric dipoles. Further, molecules contained in the sample gas are adsorbed to the surface of the electrode 12a or the electrode 12b to produce dipoles. This produces a potential difference between the electrode 12a and the electrode 12b.

The potential difference between the two electrodes is produced by a difference in interaction between the component subject to detection and the surface of each of the two electrodes. By measuring the potential difference between two electrodes paired in combinations that differ in the type, composition, surface condition, etc. of the metal, therefore, it is possible to detect whether a component subject to detection is found and the amount thereof with high precision.

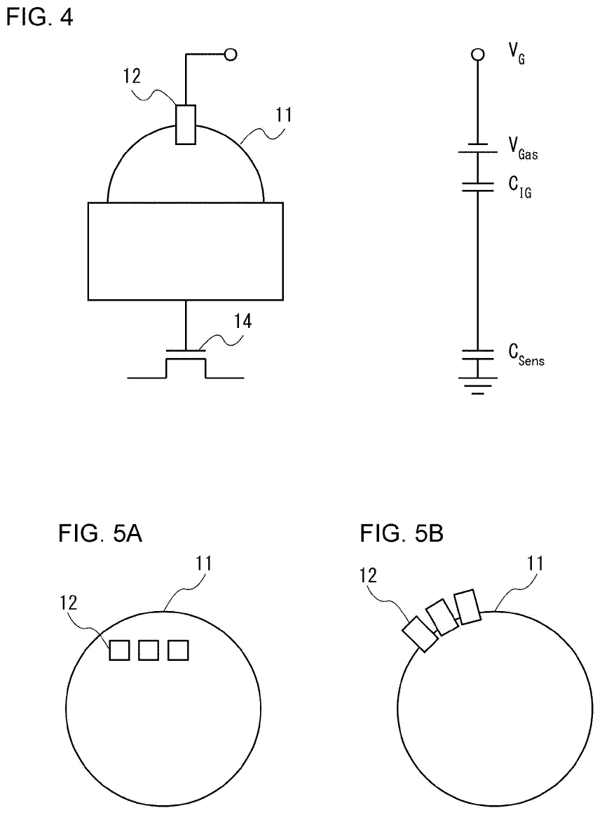

FIG. 4 shows an equivalent circuit of the sensor unit 10. Denoting the capacitance of the electric double layer as C_{IG} and the gate capacitance of the transistor 14 as C_{Sens} in this equivalent circuit,

$${V}_{IG} = \frac{{C}_{Sens} ( {V}_{Gas} + {V}_{G} )}{( {C}_{IG} + {C}_{Sens} )} \\ {V}_{Sens} = \frac{{C}_{IG} ( {V}_{Gas} + {V}_{G} )}{( {C}_{IG} + {C}_{Sens} )}$$

Therefore, a sufficient sensor response can be obtained if C_{IG}>C_{Sens}. The potential difference in this case is considered to proportional to the surface concentration of electric dipoles and not dependent on the area of the electrode.

C_{Sens} denotes the gate capacitance of the transistor 14, which is decreased when the transistor 14 is fabricated in smaller sizes. For example, the gate capacitance C_{Sens} of the transistor 14 having a gate length 40 nm, a gate width 200 nm, and a gate oxide film thickness 1.9 nm is 7.3×10⁼² fF. C_{IG} denotes the capacitance of the electric double layer of the ion conductor 11, which is proportional to the area of contact between the ion conductor 11 and the electrode 12. Given, for example, that the interlayer distance of the electric double layer is 2 nm and is constant and the ion conductor 11 and the electrode 12 is in contact in a 1 μm square, the capacitance C_{IG} of the electric double layer of the ion conductor 11 is 4.4 fF, which is sufficiently higher than the gate capacitance C_{Sens} of the transistor 14. Therefore, the area of contact between the ion conductor 11 and the electrode 12 can be reduced to a magnitude on the order of μm.

The larger the number of electrodes 12, the larger number of combinations of two electrodes for which the potential difference can be measured so that detection precision can be increased. However, the larger the number of electrodes 12, the larger the size of the sensor unit 10. To address this issue, the sensor unit 10 of the embodiment is configured such that the plurality of electrodes 12 are in contact with the common ion conductor 11. For example, he sensor unit 10 may be manufactured by coating the surface of a large number of electrodes 12 integrated on a substrate with ejected droplets of the ion conductor 11. This can realize the microscale sensor unit 10 having a large number of electrodes 12 so that the size is prevented from increasing and, at the same time, detection precision can be increased.

FIG. 5A and FIG. 5B show an exemplary configuration of the ion conductor 11 and the electrode 12 of the sensor unit 10. FIG. 5A shows an example of coating the surface of three or more electrodes 12 integrated on a substrate with the ion conductor 11 ejected by ink jetting or the like. With this configuration, the entirety of the surface of all electrodes 12 can be configured to be in contact with the ion conductor 11 so that reproducibility of measurement can be increased, and dependence on individual products caused by manufacturing errors, etc. can be reduced. This can improve detection precision of the sensor unit 10. In this case, the fluid sample is indirectly in contact with the electrode 12 via the ion conductor 11. FIG. 5B shows an example in which three or more electrodes 12 are arranged around the ion conductor 11. This configuration also allows a large number of electrodes 12 to be in contact with the common ion conductor 11 so that the size of the sensor unit 10 is prevented from increasing and, at the same time, detection precision can be improved. In this case, the fluid sample is indirectly in contact, via the ion conductor 11, with the portion of the electrode 12 covered by the ion conductor 11, and the fluid sample is directly in contact with the portion not covered by the ion conductor 11.

The sample may be a gas, a liquid, a gel, etc. The sample may be introduced on the surface of the ion conductor 11 or three or more electrodes 12 from a flow channel (not shown). The sample may be blown onto the surface of the ion conductor 11 or three more electrodes 12.

The ion conductor 11 may be an arbitrary ion liquid. The ion conductor 11 may be an arbitrary ion gel.

Three or more electrodes 12 are provided such that the type, composition, or surface condition of the metal constituting the electrode differ from each other. The electrode 12 may be integrated on a substrate, etc. by an arbitrary integration technology. The surface of the electrode 12 may be chemically modified by a functional group such as an organic group or plated by another metal. Atoms or molecules of another element may be adsorbed on the surface. In this case, the type, amount, concentration of the functional group introduced on the surface of a plurality of electrodes 12 may differ. The type, thickness, etc. of the metal to plate the surface of a plurality of electrodes 12 may differ. The type, amount, concentration, degree of adsorption of the chemical species adsorbed on a plurality of electrodes 12 may differ. Alternatively, the surface of the electrode 12 may be chemically or physically treated. In this case, the type and degree of chemical or physical treatment on the surface of a plurality of electrodes 12 may differ. Alternatively, the surface of the electrode 12 may be formed to be porous. In this case, a plurality of electrodes 12 may be formed to have different surface porosity.

The drying part 16 may be a desiccant such as silica gel, calcium oxide, and calcium chloride. An alternative feature for reducing moisture contained in the sample gas or the ion conductor 11 may be provided as the drying part 16 in addition to or in place of a desiccant. For example, a feature for blowing dry air onto the surface of the ion conductor 11 before measurement may be provided. As described later, reproducibility and reliability of measurement results can be increased by reducing moisture contained in the ion conductor 11 before measurement so that detection precision can be improved. The drying part 16 may be provided to reduce moisture contained in the sample gas.

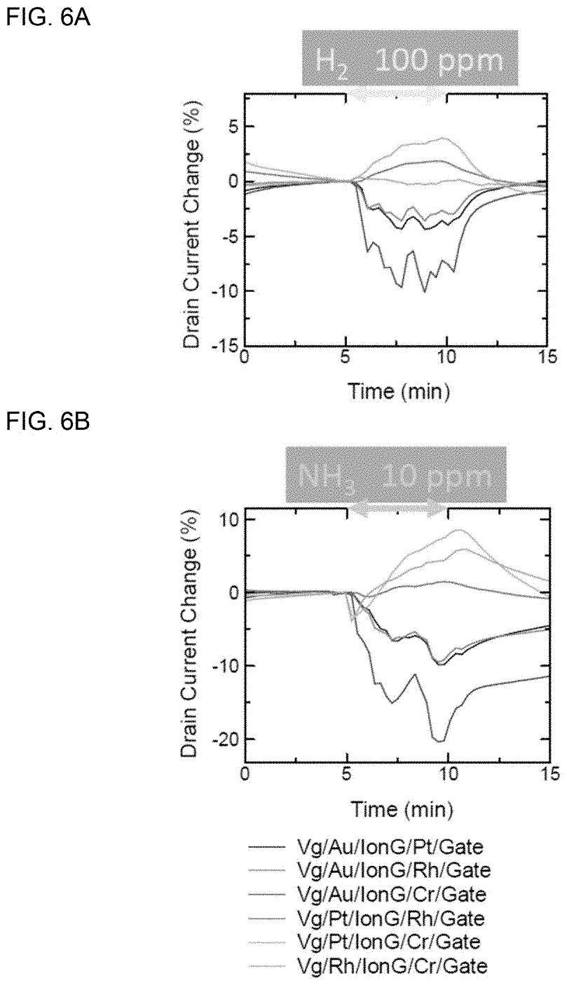

FIGS. 6A and 6B show exemplary measurement results determined by the sensor unit 10 of an exemplary embodiment. As an exemplary embodiment of the sensor unit 10 according to the embodiment, we fabricated the sensor unit 10 provided with four electrodes 12 made of four metals including gold (Au), platinum (Pt), rhodium (Rh), and chromium (Cr) and conducted experiments. The sensor unit 10 of the exemplary embodiment was not provided with the drying part 16.

FIG. 6A shows a change rate of the drain current measured by the ammeter 15 in six types of combinations each comprising two electrodes when a sample gas containing 100 ppm of hydrogen was introduced in the sensor unit 10 of the exemplary embodiment. FIG. 6B shows a change rate of the drain current measured by the ammeter 15 in six types of combinations each comprising two electrodes when a sample gas containing 10 ppm of ammonia was introduced in the sensor unit 10 of the exemplary embodiment. In either case, a gas that does not contain hydrogen or ammonia was introduced for five minutes since the start of measurement, a sample gas that contains hydrogen or ammonia was introduced from five minutes after until ten minutes after, and a gas that does not contain hydrogen or ammonia is introduced again after ten minutes. Measurement for six types of combinations of two electrodes were conducted collectively by changing the combination of two electrodes by means of the switch matrix 13. When a sample gas containing hydrogen or ammonia was introduced, the six types of combinations of two electrodes exhibited different time variations in the drain current. In any of the combinations of two electrodes, the drain current exhibited a change immediately when a sample gas containing hydrogen or ammonia was introduced. When introduction of the sample gas was stopped, the drain current returned to the original value gradually.

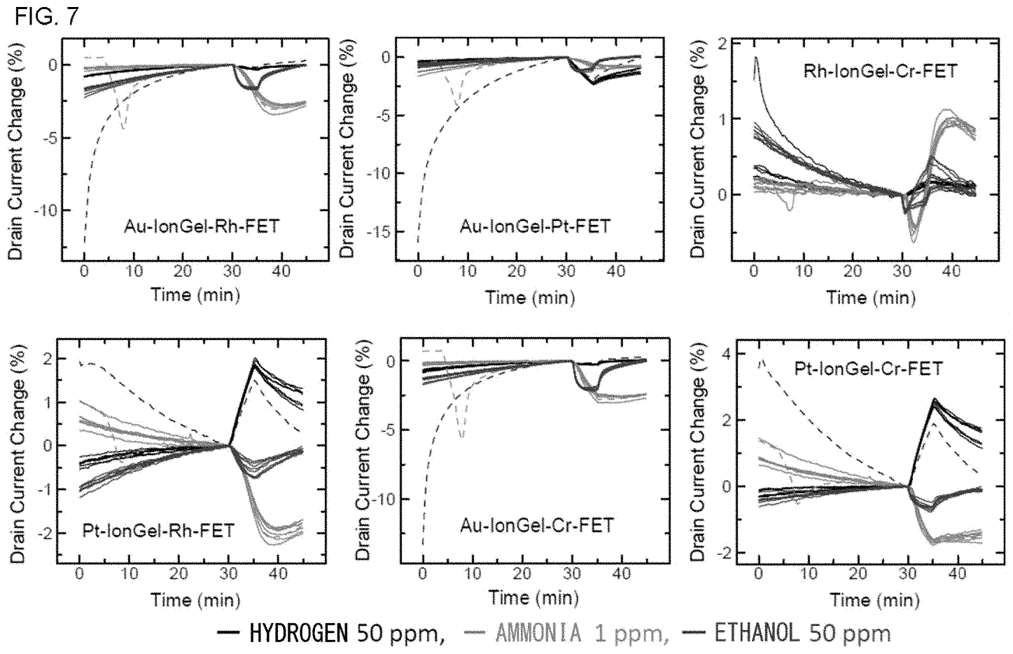

FIG. 7 shows exemplary measurement results determined by the sensor unit 10 of the exemplary embodiment. A sample gas containing 50 ppm of hydrogen, a sample gas containing 1 ppm of ammonia, and a sample gas containing 50 pm of ethanol were subject to measurement eight times each. For each sample gas, substantially the same time variation was observed in the eight measurements, demonstrating high reproducibility. The measurement results denoted by the broken lines indicate a behavior radically different from the other measurement results but are those of the first instance of measurement without exceptions, and the behavior is ascribable to moisture contained in the ion conductor 11. By providing the drying part 16 in the sensor unit 10, moisture contained in the ion conductor 11 can be reduced so that reproducibility of measurement is increased and detection precision can be improved.

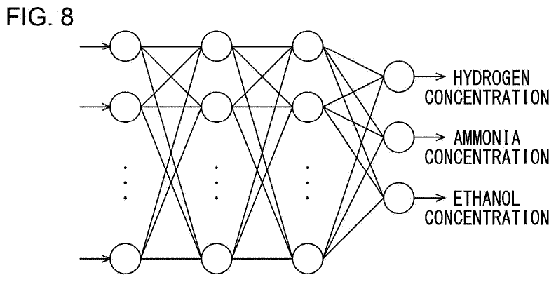

FIG. 8 shows an exemplary configuration of a component estimator for estimating the concentration of each component by referring to the measurement result determined by the sensor unit 10 of the exemplary embodiment. The component estimator may be realized by arbitrary artificial intelligence. In the exemplary embodiment, the component estimator is configured as a neural network having two hidden layers. A total of 60 measurement results, and, more specifically, 10 measurement results for each of the six types of combinations of two electrodes, are input to the input layer of the neural network, and the output layer outputs the concentration of each of hydrogen ammonia, and ethanol. A large number of mixed gases, for which the concentration of hydrogen, ammonia, and ethanol is known, are used as training samples to conduct measurement by the sensor unit 10 of the exemplary embodiment. The component estimator is trained by adjusting the weights between the neurons such that, when the measured drain current value is input to the input layer, the output layer outputs the concentration of hydrogen, ammonia, and ethanol contained in the training samples.

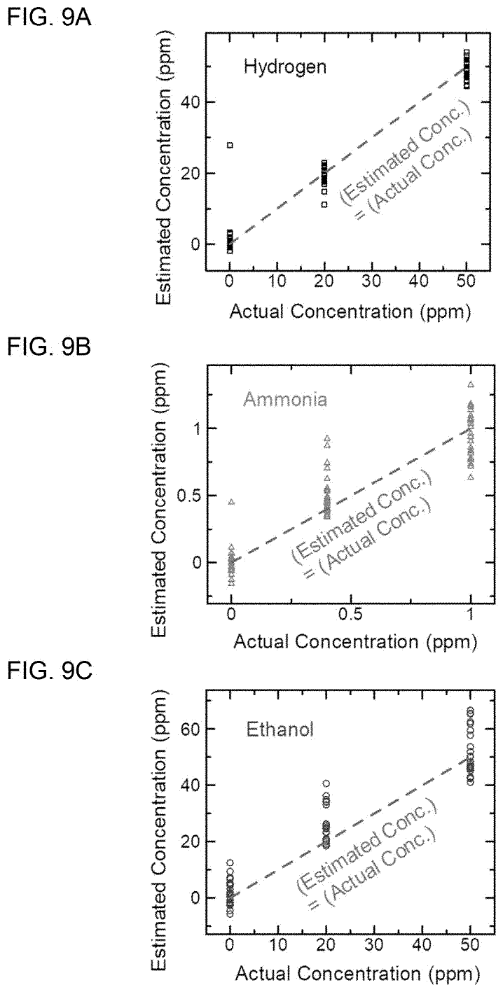

FIG. 9A to FIG. 9C show results determined by estimating the concentration of components subject to detection included in the sample gases by using the component estimator that has been trained. Mixed gases containing hydrogen, ammonia, and ethanol were used samples to conduct measurement by the sensor unit 10 of the exemplary embodiment. The measurement results were input to the input layer of the neural network that has been trained to estimate the concentration of hydrogen, ammonia, and ethanol. FIG. 9A shows a result of estimation of the concentration of hydrogen, FIG. 9B shows a result of estimation of the concentration of ammonia, and FIG. 9C shows a result of estimation of the concentration of ethanol. It is demonstrated that the component estimator can estimate the concentration close to the actual concentration for all components subject to detection. It is expected that detection precision is further improved if the number of electrodes is increased.

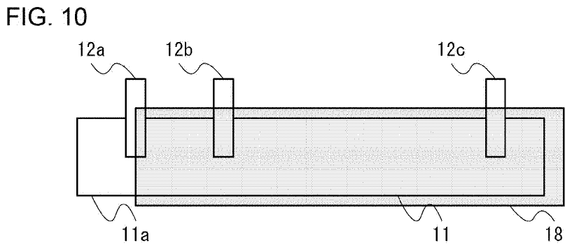

FIG. 10 shows another exemplary configuration of the ion conductor 11 and the electrodes 12 of the sensor unit 10 according to the embodiment. The electrodes 12a-12c are provided to be in contact with the ion conductor 11 at positions of different distances from a contact portion 11a where the ion conductor 11 and the sample gas are in contact. More specifically, the electrode 12a is provided to be in contact with the ion conductor 11 in the vicinity of the contact portion 11a, the electrode 12b is provided to be in contact with the ion conductor 11 at a position distanced from the contact portion 11b, and the electrode 12c is provided to be in contact with the ion conductor 11 at a position further distanced from the contact portion 11a.

The time variation in the concentration of each component contained in the sample gas at the positions of the respective electrodes differ in accordance with the coefficient of diffusion of each component in the ion conductor 11 and with the distance from the contact portion 11a. This results in time variations in the potential difference that differ between the sets of two electrodes of the electrodes 12a-12c. By measuring the time variation in the potential difference between two electrodes respectively, therefore, it is possible to detect whether a component subject to detection is found and the amount thereof with high sensitivity and high precision. In the illustrated case, the type, composition, or surface condition of the metal constituting the respective electrodes 12 may be the same. The types of combinations of two electrodes subject to measurement of a potential difference can be increased even if the type, composition, or surface condition of the metal forming the electrodes 12 are the same so that the manufacturing cost of the sensor unit 10 is prevented from increasing, and, at the same time, detection precision can be increased.

It is preferred to select, as the ion conductor 11, an ion liquid or an ion gel for which the coefficient of diffusion of a component subject to detection and the coefficient of diffusion of a component included in the sample gas other than the component subject to detection differ. This can increase detection precision of the component subject to detection.

It is preferred to ensure that the sample gas is not in contact with a position other than the contact portion 11a, and, in particular, the ion conductor 11 in a portion including the position of contact with the electrode 12. For example, a cap 18 that covers a portion of the ion conductor 11 other than the contact portion 11a may be provided as shown in the figure. Alternatively, a partition wall that separates the space around the contact portion 11a from the space around the portion other than the contact portion 11a may be provided. This inhibits a component of the sample gas from being mixed in the ion conductor 11 in a portion other than the contact portion 11a so that precision of detection of the component subject to detection can be increased.

It is preferred that the drying part 16 be provided in the illustrated example, too. The drying part 16 may be provided in the vicinity of the contact portion 11a. When the cap 18 is provided, the drying part 16 need not be provided.

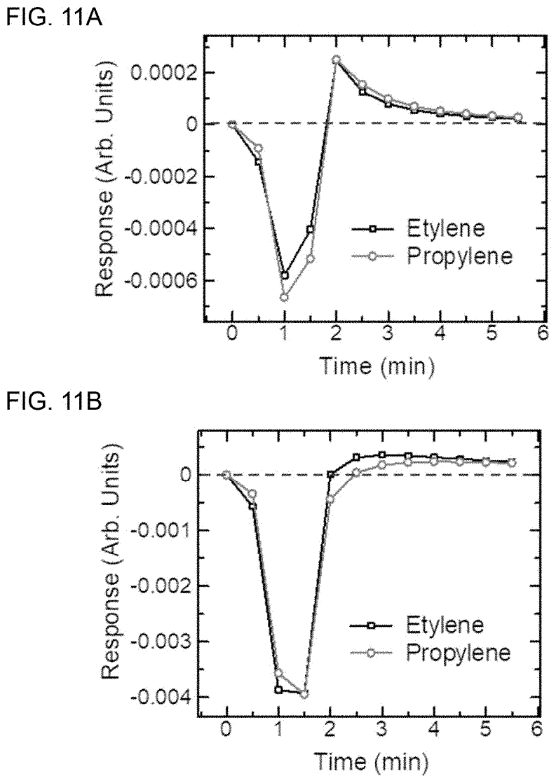

FIG. 11A and FIG. 11B show a result of simulation of the response in the sensor unit 10 of the exemplary embodiment. [emim(1-ethyl-3-methylimidazolium)][Tf₂N(bis(trifluoromethanesulfonyl)imide] was used as the ion conductor 11. The portion of 100 μm from one end of the ion conductor 11 of 2100 μm was configured as the contact portion 11a. The portion elsewhere is covered by the cap 18. The electrode 12a was provided at a position of 100 μm from one end of the ion conductor 11. The electrode 12b was provided at a position distanced from the electrode 12a by 100 μm, and the electrode 12c was provided at a position distanced from the electrode 12b by 600 μm. A sample gas containing 100 ppm of ethylene and a sample gas containing 100 ppm of propylene were blown onto the contact portion 11a for one minute each. The time variation in the potential difference between the electrode 12a and the electrode 12b and the time variation in the potential difference between the electrode 12a and the electrode 12c were measured. The coefficient of diffusion of ethylene in [emim][Tf₂N] is 0.51×10⁻⁹ m²/s, and the coefficient of diffusion of propylene is 0.33×10⁻⁹ m²/s.

FIG. 11A shows the time variation in the potential difference between the electrode 12a and the electrode 12b, and FIG. 11B shows the time variation in the potential difference between the electrode 12a and the electrode 12c. FIG. 11A reveals that there is no significant difference between the sample containing ethylene and the sample containing propylene, but FIG. 11B reveals that a difference of about 30 seconds is created between the sample containing ethylene and the sample containing propylene in the time elapsed until the response changes from negative to positive. By suitably selecting the distance between the contact portion 11a and the electrode 12, therefore, it is possible to detect ethylene and propylene contained in the sample gas in distinction from each other.

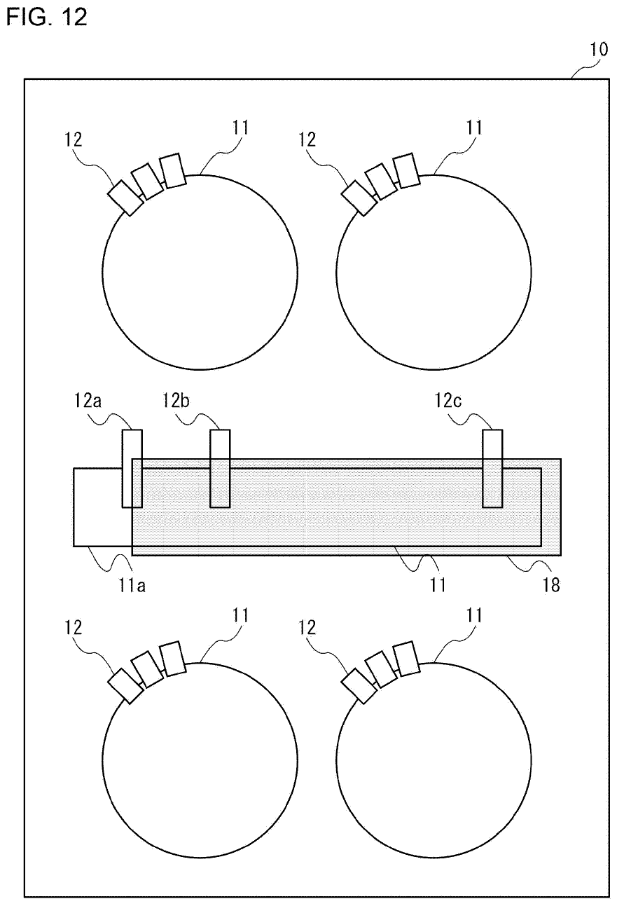

FIG. 12 shows another example of the ion conductor 11 and the electrodes 12 of the sensor unit 10 according to the exemplary embodiment. In the illustrated example, the ion conductor 11 and the electrodes 12 shown in FIG. 5A and the ion conductor 11 and the electrodes 12 shown in FIG. 9A to FIG. 9C are co-located. The ion conductors 11 may be an ion liquid or an ion gel of the same type or ion liquids or ion gels of different types. According to the illustrated example, the type of combinations of two electrodes subject to measurement of a potential difference can be increased by increasing the types of ion conductors 11 or the types of detection schemes and without increasing the types of substance, composition, surface condition, etc. of the metal constituting the electrode 12. Accordingly, the manufacturing cost of the sensor unit 10 is prevented from being increased, and, at the same time, detection precision can be increased.

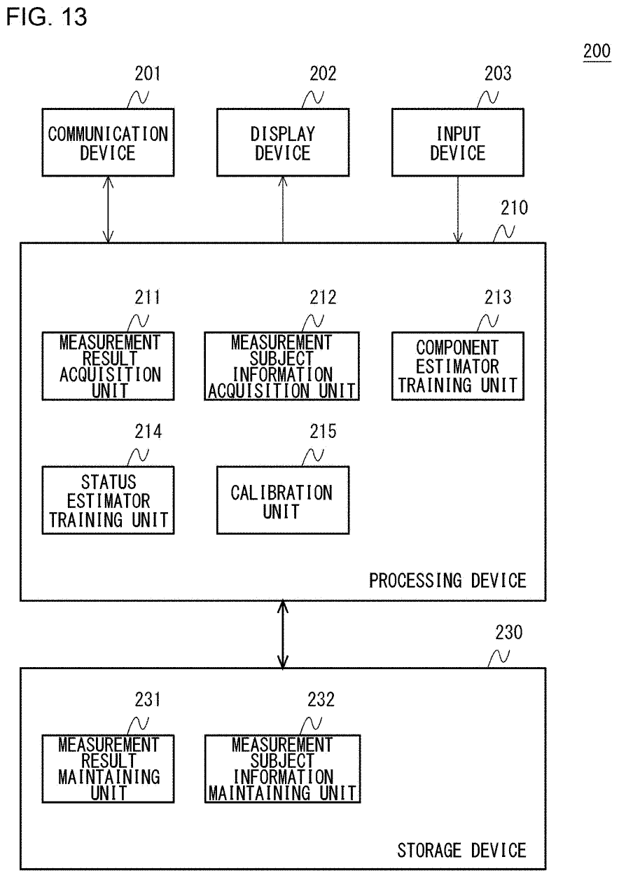

FIG. 13 shows a configuration of a learning device 200 according to the embodiment. The learning device 200 is provided with a communication device 201, a display device 202, an input device 203, a storage device 230, and a processing device 210. The learning device 200 may be a server device or a device such as a personal computer, or a mobile terminal such as a cellular phone terminal, a smartphone, and a tablet terminal.

The communication device 201 controls communication with other devices. The communication device 201 may communicate with other devices by using an arbitrary wire or wireless communication scheme. The display device 202 displays a screen generated by the processing device 210. The display device 202 may be a liquid crystal display device, an organic EL display device, etc. The input device 203 transmits an input for instruction provided by the user of the learning device 200 to the processing device 210. The input device 203 may be a mouse, a keyboard, a touchpad, etc. The display device 202 and the input device 203 may be embodied by a touch panel.

The storage device 230 stores programs, data, etc. used by the processing device 210. The storage device 230 may be a semiconductor memory, a hard disk, etc. The storage device 230 stores a measurement result maintaining unit 231 and a measurement subject information maintaining unit 232.

The processing device 210 is provided with a measurement result acquisition unit 211, a measurement subject information acquisition unit 212, a component estimator training unit 213, a status estimator training unit 214, and a calibration unit 215. The features are implemented in hardware such as a central processing unit (CPU), a memory, or other large scale integration (LSI), of any computer and in software such as a program loaded into a memory. The figure depicts functional blocks implemented by the cooperation of these elements. Therefore, it will be understood by those skilled in the art that the functional blocks may be implemented in a variety of manners by hardware only or by a combination of hardware and software.

The measurement result acquisition unit 211 acquires measurement results from the detection device 100 and stores the measurement results in the measurement result maintaining unit 231. The measurement subject information acquisition unit 212 acquires information relating to the sample subject to measurement from the detection device 100 and stores the information in the measurement subject information maintaining unit 232.

The component estimator training unit 213 trains the component estimator by using the measurement results stored in the measurement result maintaining unit 231 as training data. As described above, the component estimator may be configured as a neural network. In this case, the component estimator training unit 213 adjusts the weights between neurons such that, when the measurement result from a training sample for which a component is known is input to the input layer, the output layer outputs whether a component subject to detection included in the training sample is found or the amount thereof.

The component estimator may be configured to calculate the amount of a component subject to detection included in the sample according to a mathematical expression using the measurement result. In this case, the component estimator training unit 213 adjusts coefficients, etc., in the mathematical expression such that, when the measurement result from a training sample for which a component is known is input to the mathematical expression, the amount of the component subject to detection included in the training sample is calculated. The mathematical expression may be a linear polynomial expression in which each of the current values measured in the respective electrodes is multiplied by a coefficient. In this case, the component estimator training unit 213 may adjust each coefficient of the linear polynomial expression by multiple linear regression analysis.

The status estimator training unit 214 trains the status estimator for estimating the status of the sample from the measurement result, by using the measurement result stored in the measurement result maintaining unit 231 and the information relating to the sample subject to measurement stored in the measurement subject information maintaining unit 232. The status estimator may be used to estimate the health condition of a subject person, a disease that the subject person is affected with, etc., by referring to, for example, the measurement result yielded by using the air exhaled by the subject person as a sample. The status estimator training unit 214 may train the status estimator by categorizing or clustering the measurement results stored in the measurement result maintaining unit 231.

The calibration unit 215 generated information for calibrating the detection device 100. In the sensor unit 10 of the detection device 100, the measurement result may depend on individual products due to minor manufacturing errors such as the composition and surface condition of the metal constituting the electrode 12, the status of contact between the electrode 12 and the ion conductor 11, etc. The calibration unit 215 compares measurement results from a plurality of detection devices 100, generates information for calibrating the measurement results, and provides the information to the detection device 100. The detection device 100 calibrates the measurement result based on the information provided from the learning device 200 before inputting the measurement result to the component estimator or the status estimator. This can cancel dependence of the sensor unit 10 on individual products and improve estimation precision. The calibration unit 215 may calibrate the component estimator or the status estimator to suit individual detection devices 100.

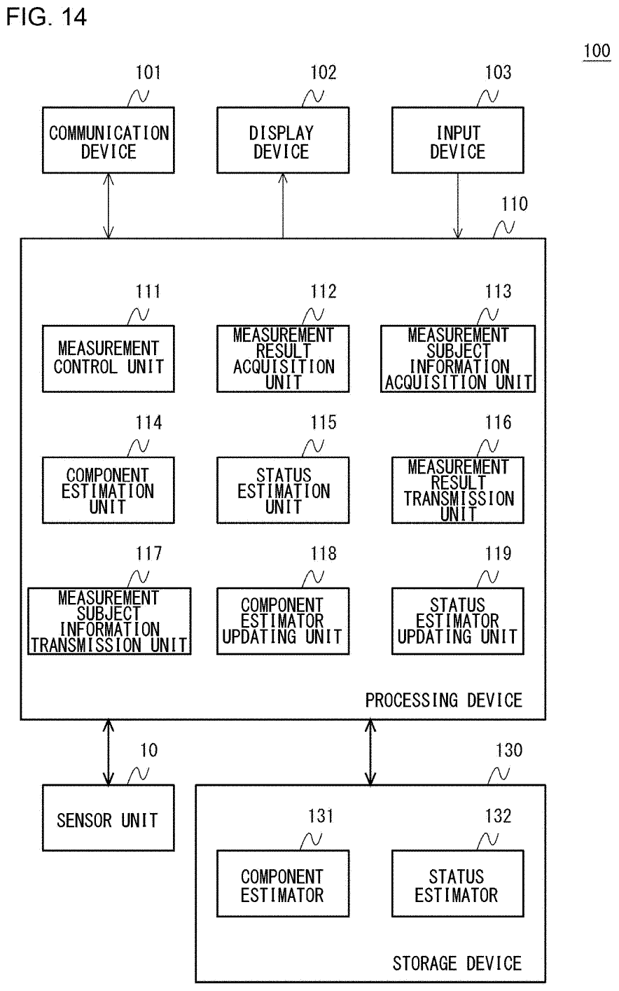

FIG. 14 shows a configuration of the detection device 100 according to the embodiment. The detection device 100 is provided with the sensor unit 10, a communication device 101, a display device 102, an input device 103, a storage device 130, and a processing device 110. The detection device 100 may be a server device or a device such as a personal computer, or a mobile terminal such as a cellular phone terminal, a smartphone, and a tablet terminal.

The communication device 101 controls communication with other devices. The communication device 101 may communicate with other devices by using an arbitrary wire or wireless communication scheme. The display device 102 displays a screen generated by the processing device 110. The display device 102 may be a liquid crystal display device, an organic EL display device, etc. The input device 103 transmits an input for instruction provided by the user of the detection device 100 to the processing device 110. The input device 103 may be a mouse, a keyboard, a touchpad, etc. The display device 102 and the input device 103 may be embodied by a touch panel.

The storage device 130 stores programs, data, etc. used by the processing device 110. The storage device 130 may be a semiconductor memory, a hard disk, etc. The storage device 130 stores a component estimator 131 and a status estimator 132.

The processing device 110 is provided with a measurement control unit 111, a measurement result acquisition unit 112, a measurement subject information acquisition unit 113, a component estimation unit 114, a status estimation unit 115, a measurement result transmission unit 116, a measurement subject information transmission unit 117, a component estimator updating unit 118, and a status estimator updating unit 119. These features can also be implemented in a variety of manners by hardware only or by a combination of hardware and software.

The measurement control unit 111 controls measurement by the sensor unit 10. The measurement control unit 111 determines a combination of two electrodes for which a potential difference is measured in accordance with the type, status, and amount of the sample, type of a component subject to detection, type and amount of a component other than the component subject to detection included in the sample, etc. The measurement control unit 111 causes the switch matrix 13 to select the two electrodes of the combination thus determined. The measurement control unit 111 causes the drying part 16 to reduce moisture contained in the ion conductor 11 and then causes the power source to apply a voltage to the measurement terminal 17 and the drain terminal of the transistor 14 and causes the ammeter 15 to measure the current value.

The measurement result acquisition unit 112 acquires the measurement result from the sensor unit 10. The measurement result acquisition unit 112 acquires time series data for the current value measured by the ammeter 15 at predetermined intervals until a predetermined time elapses since the start of measurement.

The measurement subject information acquisition unit 113 acquires information relating to the sample of measurement subject. When the sample is a gas collected from the exhaled air of a subject person, the measurement subject information acquisition unit 113 acquires information such as the health condition, age, sex, personal medical history, body temperature, time elapsed after a meal, contents of the meal via the communication device 101 or the input device 103.

The component estimation unit 114 estimates whether a component subject to detection included in the sample is found or the amount thereof based on the measurement result acquired by the measurement result acquisition unit 112. The component estimation unit 114 estimates whether a component subject to detection is found or the amount thereof by using the component estimator 131 that has been trained. When the information for calibrating the measurement result is received from the learning device 200, the component estimation unit 114 calibrates the measurement result before inputting the measurement result to the component estimator 131.

The status estimation unit 115 estimates the status of the sample based on the measurement result acquired by the measurement result acquisition unit 112. The status estimation unit 115 estimates the health condition of the subject, a disease that the subject is affected with, etc. by using the status estimator 132 that has been trained. When the information for calibrating the measurement result is received from the learning device 200, the status estimation unit 115 calibrates the measurement result before inputting the measurement result to the status estimator 132.

The measurement result transmission unit 116 transmits the measurement result acquired by the measurement result acquisition unit 112 to the learning device 200. The measurement subject information transmission unit 117 transmits the measurement subject information acquired from the measurement subject information acquisition unit 113 to the learning device 200. These items of information are used to train the component estimator 131 and the status estimator 132 further in the learning device 200.

The component estimator updating unit 118 acquires the component estimator from the learning device 200 and updates the component estimator 131 stored in the storage device 130. The status estimator updating unit 119 acquires the status estimator from the learning device 200 and updates the status estimator 132 stored in the storage device 130. In this way, estimation precision can be improved.

The detection device 100 may be packaged in an integrated circuit. For example, a part or the entirety of the sensor unit 10 and the processing device 120 may be packaged on a single chip. In this way, the size of the detection device 100 can be reduced so that the detection device 100 can be built in various equipment easily. In this case, the component estimator 131 may be configured to calculate the amount of a component subject to detection included in the sample according to a mathematical expression using the measurement result. This can suppress the processing load in the component estimation unit 114 so that the size, weight, and manufacturing cost of the detection device 100 can be further reduced, and, ultimately, the size, weight, and manufacturing cost of the equipment in which the detection device 100 is built can be reduced.

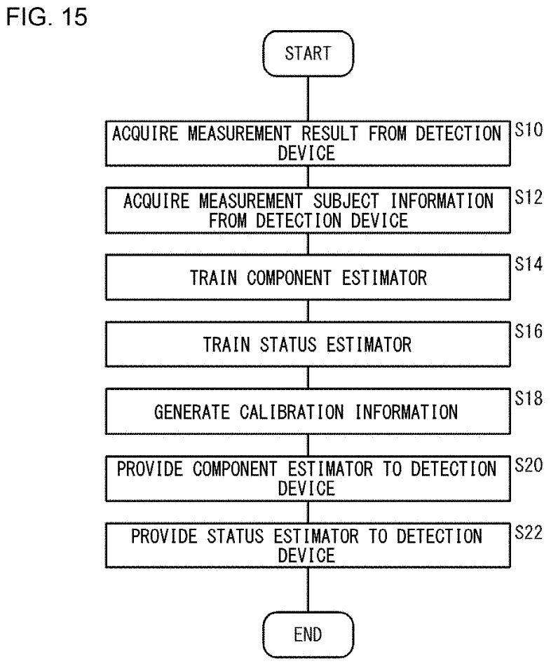

FIG. 15 is a flowchart showing a sequence of steps of the learning method according to the embodiment. The measurement result acquisition unit 211 of the learning device 200 acquires the measurement result from the detection device 100 (S10). The measurement subject information acquisition unit 212 acquires the information relating to the sample subject to measurement from the detection device 100 (S12). The component estimator training unit 213 trains the component estimator by using the measurement result as training data (S14). The status estimator training unit 214 trains the status estimator by using the measurement result and the information relating to the sample subject to measurement (S16). The calibration unit 215 generates information for calibrating the detection device 100 (S18). The learning device 200 provides the component estimator that has been trained to the detection device 100 (S20). The learning device 200 provides the status estimator that has been trained to the detection device 100 (S22).

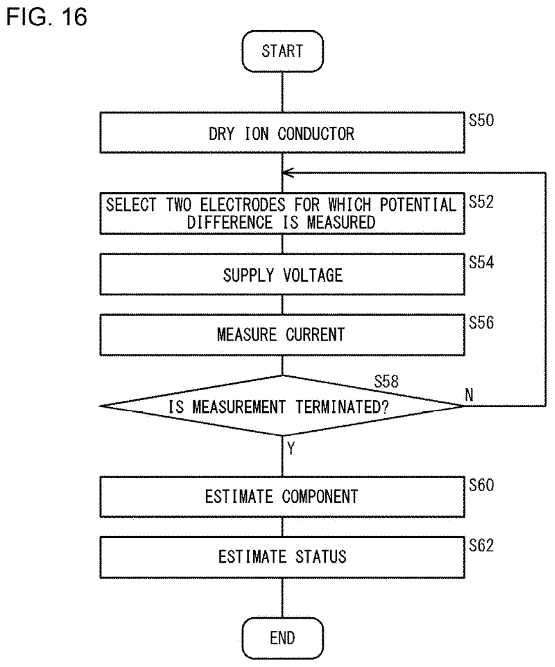

FIG. 16 is a flowchart showing a sequence of steps of the detection method according to the embodiment. The measurement control unit 111 of the detection device 100 causes the drying part 16 to dry the ion conductor 11 (S50). The switch matrix 13 selects two electrodes for which a potential difference is measured (S52). The measurement control unit 111 supplies a voltage from the power source (S54) to cause the ammeter 15 to measure the current value (S56). The measurement control unit 111 repeats S52 through S56 until the measurement is completed (N in S58). When the measurement for a predetermined period of time is completed for all combinations of two electrodes for which a potential difference is measured (Y in S58), the component estimation unit 114 estimates whether a component subject to detection included in the sample is found or the amount thereof based on the measurement result (S60), and the status estimation unit 115 estimates the status of the sample based on the measurement result (S62).

Described above is an explanation based on an exemplary embodiment. The embodiment is illustrative, and it will be understood by those skilled in the art that various modifications to constituting elements and processes could be developed and that such modifications are also within the scope of the present disclosure.

In the embodiment, the status estimator is described, by way of example, as using a component included in exhaled air in a small amount as a biomarker. However, the technology of the present disclosure is applicable to estimation of the status of food or drink by referring to a component subject to detection included in a gas produced from food or drink or to estimation of the operating condition of a mobile object or a plant by referring to a component subject to detection included in a discharged gas discharged from the mobile object or the plant.

What is claimed is:

1. A detection device comprising:
an ion conductor;
three or more electrodes that are in contact with the ion conductor; and
a measurement unit that measures a potential difference between two electrodes when a fluid sample is in contact with the ion conductor or the electrode, the two electrodes being selected from the three or more electrodes in a plurality of combinations.

2. The detection device according to claim 1, wherein
at least two electrodes are in contact with a common ion conductor.

3. The detection device according to claim 1, wherein
the three or more electrodes differ in at least one of:
a type, composition, or surface condition of a metal constituting the electrode;
a type of the ion conductor that the electrode is in contact with; and
a distance from a position of contact between the sample and the ion conductor to a position of contact between the electrode and the ion conductor.

4. The detection device according to claim 1, wherein
a combination of two electrodes for which a potential difference is measured is selected based on at least one of: a type and amount of a component subject to detection included in the sample; and a type and amount of a component that could be included in the sample other than the component subject to detection.

5. The detection device according to claim 1, further comprising:
a drying part for reducing moisture contained in the sample or the ion conductor.

6. The detection device according to claim 1, wherein
a portion of the ion conductor including a position of contact with the electrode is configured not to be in contact with the sample.

7. The detection device according to claim 1, further comprising:
an estimation unit that estimates whether a component included in the sample is found or an amount thereof based on a potential difference, measured by the measurement unit, between two electrodes paired in a plurality of combinations.

8. The detection device according to claim 7, wherein
the estimation unit estimates whether a component included in the sample is found or an amount thereof by using an estimator trained by using, as a training sample, a fluid for which a component is known and using, as training data, data indicating potential differences, measured by the measurement unit, of two electrodes paired in a plurality of combinations.

9. The detection device according to claim 8, wherein
the estimator inputs time series data for potential differences, measured by the measurement unit, between two electrodes paired in a plurality of combinations to an input layer and outputs whether a component included in the sample is found or an amount thereof from an output layer.

10. A detection method comprising:
measuring, when an ion conductor or three or more electrodes in contact with the ion conductor is in contact with a sample of fluid, a potential difference between two electrodes selected from the three or more electrodes, measurements being made a plurality of times for different combinations of two electrodes.

11. The detection method according to claim 10, further comprising:
estimating whether a component included in the sample is found or an amount thereof based on potential differences between two electrodes paired in a plurality of combinations.

12. The detection method according to claim 10, further comprising:
reducing moisture contained in the ion conductor before measuring potential differences between two electrodes paired in a plurality of combinations.

13. A learning device comprising:
a training data acquisition unit that acquires, as training data, data indicating potential differences, measured by the measurement unit, between two electrodes paired in a plurality of combinations by using a fluid for which a component is known as a training sample, from the detection device according to claim 1; and
a training unit that trains, by using the training data acquired by the training data acquisition unit, an estimator for estimating whether a component included in a sample of fluid is found or an amount thereof.

14. The learning device according to claim 13, wherein
the estimator is comprised of a neural network, and
the training unit adjusts an intermediate layer of the neural network such that, when the training data is input to an input layer of the neural network, an output layer of the neural network outputs whether a component included in the training sample is found or an amount thereof.

15. A learning device comprising:
a training data acquisition unit that acquires, as training data, information relating to each of a plurality of samples and data indicating potential differences, measured by the measurement unit in the samples, between two electrodes paired in a plurality of combinations; and
a training unit that categorizes or clusters the training data acquired by the training data acquisition unit.

16. A method of manufacturing the detection device according to claim 1, comprising:
determining a type, composition, or surface condition of a metal constituting the three or more electrodes based on at least one of: a type and amount of a component subject to detection included in the sample; and a type and amount of a component that could be included in the sample other than the component subject to detection; and
providing the three or more electrodes constituted by a metal of a type, composition, or surface condition determined so as to be in contact with the ion conductor.

17. The method according to claim 16, wherein
the three or more electrodes are provided so as to be in contact with the ion conductor by coating a surface of the three or more electrodes with droplets of the ion conductor.
